(19) United States
(12) Patent Application Publication
(10) Pub. No.: US 2026/0208178 A1
(43) Pub. Date: Jul. 23, 2026

(54) ALKANE DEHYDROGENATION CATALYST

(71) Applicant: SABIC GLOBAL TECHNOLOGIES B.V., Bergen op Zoom (NL)

(72) Inventors: Biju Maippan Devassy, Bangalore (IN); Vinod Sankaran Nair, Bangalore (IN); Velayutham Saravanan, Hosur (IN); Nigit Jose Meleppuram, Bangalore (IN); Ritesh Nandy, Bengaluru (IN); Mahabala PS, Bangalore (IN); Vishal Patrick, Bengaluru (IN)

(21) Appl. No.: 19/140,927

(22) PCT Filed: Dec. 18, 2023
(86) PCT No.: PCT/EP2023/086281

§ 371 Date: Jun. 18, 2025

(30) Foreign Application Priority Data
Dec. 22, 2022 (IN) 202241074442

(51) Int. Cl. B01J 37/08; B01J 37/00; C07C 5/333

(52) U.S. Cl. CPC B01J 37/08; B01J 37/0009; C07C 5/3332; C07C 2521/04; C07C 2523/04; C07C 2523/26

(57) ABSTRACT

Methods of preparing alkane dehydrogenation catalysts that include 10 wt. % to 40 wt. % chromium(III) oxide (Cr₂O₃), 0.1 wt. % to 5 wt. % alkali metal oxide, and 60 wt. % to 90 wt. % alumina Al2O3 are described. A method can include mixing a solid aluminum hydroxide source with a water insoluble chromium(III) oxide source, an alkali metal oxide source, and an aqueous non-metal acid to produce a catalyst precursor. The catalyst precursor can be dried and calcined at 700° C. to 1000° C. to produce the catalysts of the present invention.

### CROSS REFERENCE TO RELATED APPLICATIONS

This application claims priority to and the benefit of the filing date of European Patent Application No. 202241074442, filed Dec. 22, 2022, which is incorporated herein by reference in its entirety.

### FIELD OF INVENTION

The present invention generally relates to methods to prepare alkane dehydrogenation catalysts using a water insoluble chromium(III) oxide source, a solid aluminum hydroxide, and an alkali metal oxide source.

### BACKGROUND OF THE INVENTION

Alkane dehydrogenation is a recognized process for production of a variety of useful hydrocarbon products, such as isobutylene for conversion to MTBE, isooctane, and alkylates for supplementing and enriching gasolines. There are several current catalytic processes useful for catalytic dehydrogenation of light alkanes, including the Süd-Chemie CATOFIN® process, UOP's Oleflex® process, Phillips' Star™ process, and the Snamprogetti-Yarsintee process. The catalysts that are used in these processes are manufactured from two different groups of materials. The Süd-Chemie CATOFIN® process and the Snamprogetti-Yarsintee process use chromia-alumina catalysts. In contrast, the catalysts for the UOP and Phillips processes comprise precious metal(s) on support catalysts. Chromia-alumina dehydrogenation catalyst technology has been in use for many decades. With dehydrogenation catalysts used for the above processes, stability of the catalyst plays an important role in the overall efficiency of the dehydrogenation process. Because of the extreme temperature ranges at which the catalytic dehydrogenation procedure is conducted, the life expectancy of the catalyst often is limited. Thus, improving the stability of the catalyst translates into longer catalyst life or improved catalyst selectivity over the catalyst life, allowing for better catalyst utilization, which ultimately results in lower consumption of the catalyst during the dehydrogenation process.

There are a number of different types of alumina that are available for use as the support for dehydrogenation catalysts. However, mid to high surface area gamma alumina has consistently been the preferred choice as the carrier for such catalysts. Chromium (III) oxide supported on eta-alumina may be highly stable compared to chromia supported on gamma-alumina.

Current chromia-alumina dehydrogenation catalysts are produced by the impregnation of an aluminum carrier with a high-concentration chromic acid solution, which contains primarily hexavalent chromium (chromium (VI)), which is toxic and carcinogenic, and therefore highly undesirable for use on an industrial scale. One method of overcoming the use of hexavalent chromium in catalyst preparation involves impregnating an aluminum carrier with a water soluble chromium(III) salt, such as an aqueous solution of chromium nitrate. This method requires multiple impregnation steps to arrive at a desirable chromium oxide content. Each impregnation step would require intermediate drying and calcination, and so such a multi-impregnation method would be time- and labor-intensive, and cost prohibitive relative to conventional preparation procedures involving chromium (VI)-containing materials.

Attempts to overcome the use of hexavalent chromium have been described. For example, a Cr₂O₃/alumina carrier may be immersed with additional chromium nitrate and potassium nitrate, or alternatively, by impregnating the carrier with an aqueous solution containing chromium nitrate, sodium hydroxide, magnesium nitrate, and zirconium carbonate. These methods suffer in that the catalyst preparation involves an impregnation process followed by drying and calcination steps. Such methods suffer from being time and labor intensive, in addition to having increased costs.

Another attempt to produce a chromium based dehydrogenation catalyst includes extruding an acidic solution of chromium(III) oxide powder, activated alumina powder, calcium nitrate and potassium nitrate, followed by drying and calcining. The dried extrudates are then baked in nitrogen gas to obtain the catalyst. A catalyst formed with this process is expected to show alumina in gamma alumina form which strongly affects catalyst stability.

While many methods to produce chromium based dehydrogenation catalyst exist, they can be inefficient and expensive and/or result in catalysts that have low conversion/activity and/or limited stability.

### BRIEF SUMMARY OF THE INVENTION

A discovery has been made that provides a solution to at least one of the problems associated with methods to produce alkane dehydrogenation catalysts. In one aspect, the invention can include a method to incorporate chromium(III) oxide, at least one alkali metal oxide, an optional alkaline earth metal oxide, an optional lanthanum oxide, and aluminum hydroxide into a formed structure (e.g., an extrudate or tablet). The method eliminates the requirement for chromium (VI) containing compounds, and multi-step impregnation and/or heating procedures. Accordingly, the invention provides a simple, cost effective method for making a chromia-alumina dehydrogenation catalyst with improved stability for dehydrogenation of lower paraffins. Notably, the method does not require impregnation of additional water soluble chromium compounds after the extrudate is formed and calcined to obtain the desired amount of chromium(III) oxide in the catalyst.

In one aspect of the present invention, methods to produce alkane dehydrogenation catalysts are described. A method can include extruding and/or tableting a mixture that includes a solid aluminum hydroxide, a water insoluble chromium(III) oxide source, an alkali metal oxide source, and an aqueous non-metal acid to produce a catalyst precursor. In some aspects, the mixture can be aged at a temperature of 20° C. to 40° C. prior to extruding and/or tableting for 0.5 to 24 hours. The catalyst precursor can be dried, after which it is calcined at a temperature 700° C. to 1000° C. to produce an alkane dehydrogenation catalyst that contains 10 wt. % to 40 wt. % chromium(III) oxide (Cr₂O₃), 0.1 wt. % to 5 wt. % alkali metal oxide, and 60 wt. % to 90 wt. % alumina (Al₂O₃). In some aspects, the calcining can be performed under air. Non-limiting examples of the solid aluminum hydroxide can include crystalline aluminum trihydroxides (e.g., bayerite, nordstrandite, or a mixture thereof), crystalline aluminum oxide-hydroxides (boehmite), gelatinous aluminum hydroxides (e.g., amorphous aluminum hydroxide, pseudobohemite or a mixture thereof), or mixtures thereof. The solid aluminum hydroxide can include 90 wt. % to 100 wt. % of crystalline aluminum trihydroxides. In another aspect, the solid aluminum hydroxide can include 0 wt. % to 10 wt. % of crystalline aluminum oxide-hydroxides or gelatinous aluminum hydroxides, or a combination thereof with the balance being crystalline aluminum trihydroxides. The alkali metal oxide source can include a sodium oxide source, a lithium oxide source, a cesium oxide source, a potassium oxide source, or a mixture thereof, and is preferably sodium oxide. In some aspects, the mixture can include a lanthanide oxide source. Non-limiting examples of the lanthanide oxide source can include a lanthanum oxide source, a cerium oxide source, or a mixture thereof. In some aspects, the mixture can include an alkaline earth metal oxide source. Non-limiting examples of alkaline earth metal oxide sources can include a barium oxide (BaO) source and/or a strontium oxide (SrO) source, and the catalyst comprises BaO, SrO, or a combination thereof. In another aspect, the mixture can further include a silica source. In such aspects, the catalyst can include 0.1 wt. % to 5 wt. % silica.

The catalyst can include 10 wt. % to 40 wt. % chromium(III) oxide (Cr₂O₃), 0.1 wt. % to 5 wt. % alkali metal oxide, and 60 wt. % to 90 wt. % alumina Al₂O₃. For example, the catalyst can include 10 wt. % to 40 wt. % chromium(III) oxide (Cr₂O₃), 0.1 wt. % to 5 wt. % Na₂O, and 60 wt. % to 90 wt. % alumina Al₂O₃. As another example, the catalyst can include 10 wt. % to 40 wt. % chromium(III) oxide (Cr₂O₃), 0.1 wt. % to 5 wt. % Na₂O, 0.1 wt. % to 5 wt. % Li₂O, and 60 wt. % to 90 wt. % alumina Al₂O₃. The catalyst can include 10 wt. % to 40 wt. % chromium(III) oxide (Cr₂O₃), 0.1 wt. % to 5 wt. % alkali metal oxide, 0.1 to 20 wt. % alkaline earth metal oxide, and 60 wt. % to 90 wt. % alumina Al₂O₃. For example, the catalyst can include 10 wt. % to 40 wt. % chromium(III) oxide (Cr₂O₃), 0.1 wt. % to 5 wt. % Li₂O, 0.1 to 20 wt. % BaO, and 60 wt. % to 90 wt. % alumina Al₂O₃. In some aspects, the catalyst can include 60 wt. % to 90 wt. % Al₂O₃, 10 wt. % to 40 wt. % Cr₂O₃, 0.1 wt. % to 5 wt. % Na₂O, 0.1 wt. % to 20 wt. % alkaline earth metal oxide, and 0.01 wt. % to 3 wt. % Li₂O. For example, the catalyst can include 60 wt. % to 90 wt. % Al₂O₃, 10 wt. % to 40 wt. % Cr₂O₃, 0.01 wt. % to 5 wt. % Na₂O, 0.1 wt. % to 20 wt. % BaO, and 0.1 wt. % to 5 wt. % Li₂O. In some aspects, the catalyst can include 60 wt. % to 90 wt. % Al₂O₃, 10 wt. % to 40 wt. % Cr₂O₃, 0.1 wt. % to 5 wt. % Na₂O, and 0.1 wt. % to 5 wt. % SiO₂. In other aspects, the catalyst can include 60 wt. % to 95 wt. % Al₂O₃, 5 wt. % to 40 wt. % Cr₂O₃, 0.1 wt. % to 5 wt. % Na₂O, and 0.1 wt. % to 20 wt. % lanthanide oxide, preferably 0.1 wt. % to 5 wt. % lanthanide oxide. For example, the catalyst can include 60 wt. % to 95 wt. % Al₂O₃, 5 wt. % to 40 wt. % Cr₂O₃, 0.1 wt. % to 5 wt. % Na₂O, and 0.1 wt. % to 20 wt. % La₂O₃. In one aspect, the catalyst can include 60 wt. % to 95 wt. % Al₂O₃, 5 wt. % to 40 wt. % Cr₂O₃, 0.1 wt. % to 5 wt. % Na₂O, and 0.1 wt. % to 20 wt. % lanthanide oxide, preferably 0.1 wt. % to 5 wt. % lanthanide oxide, and 0.1 wt. % to 20 wt. % alkaline earth metal oxide. For example, the catalyst can include 60 wt. % to 95 wt. % Al₂O₃, 5 wt. % to 40 wt. % Cr₂O₃, 0.1 wt. % to 5 wt. % Na₂O, and 0.1 wt. % to 20 wt. % La₂O₃, preferably 0.1 wt. % to 5 wt. % La₂O₃, and 0.1 wt. % to 20 wt. % BaO, preferably 0.1 wt. % to 5 wt. %, more preferably 0.1 wt. % to 3 wt. % BaO.

In another aspect of the present invention, methods to dehydrogenate alkanes are described. A method can include contacting a catalyst made by any one of the methods of the present invention with an alkane to produce dehydrogenated paraffins (alkenes, preferably isobutylene from isobutane).

The following includes definitions of various terms and phrases used throughout this specification.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %”, “vol. %”, or “mol. %” refer to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, include any measurable decrease or complete inhibition to achieve a desired result.

The term “effective,” as that term is used in the specification and/or claims, means adequate to accomplish a desired, expected, or intended result.

The terms “a mixture thereof” and “a combination thereof” or any variation of these terms, when used in the claims and/or the specification in conjunction with a list of components, mean any combination of two or more of the listed components, including such combinations in which one or more of the other listed components are absent therefrom.

The use of the words “a” or “an” when used with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The methods of the present invention can “comprise,” “consist essentially of,” or “consist of” particular ingredients, components, compositions, etc. disclosed throughout the specification. With respect to the transitional phrase “consisting essentially of,” in one non-limiting aspect, a basic and novel characteristic of the methods of the present invention are their abilities to make a chromium/aluminum dehydrogenation catalyst in a cost and energy efficient manner.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

For a more complete understanding, reference is now made to the following descriptions taken in conjunction with the accompanying drawings, in which:

FIG. 1 illustrates a system to dehydrogenate alkanes, according to embodiments of the invention.

FIG. 2 depicts powder X-ray diffraction patterns of catalyst of the present invention.

While the invention is susceptible to various modifications and alternative forms, specific embodiments thereof are shown by way of example in the drawings. The drawings may not be to scale.

### DETAILED DESCRIPTION OF THE INVENTION

A discovery has been made that provides a solution to at least one of the problems associated with making alkane dehydrogenation catalysts. In one aspect of the invention, a method to produce an alkane dehydrogenation catalyst can include mixing at room temperature (e.g., 20° C. to 70° C.), a solid aluminum hydroxide and a water-insoluble chromium(III) oxide source with an alkali metal oxide source, optionally a silica source, optionally a lanthanide oxide source, optionally an alkaline earth metal source, an aqueous non-metal acid, and water to form a formable mixture. The formable mixture can be formed (e.g., extruded and/or tableted) into extrudates or tablets to the catalyst precursor. The formable mixture can be aged at 20° C. to 40° C. for 0.5 to 24 hours. The catalyst precursor can be dried and calcined to produce the alkane dehydrogenation catalyst of the present invention. An advantage of the method is that it provides a simple, cost effective method for making a chromia-alumina dehydrogenation catalyst by using a water-insoluble chromium(III) oxide source instead of chromium (VI) containing materials in combination with an alkali metal oxide source. The catalysts of the present invention have better stability for dehydrogenation of lower paraffins. While not wishing to be bound by any theory, it is believed that presence of certain alkali metal oxides and/or alkaline earth metal oxides can inhibit phase transformation within the alumina to provide a strong and thermally stable catalyst.

### A. Method of Making a Paraffin Dehydrogenation Catalyst.

Solid aluminum hydroxide and the chromium(III) oxide source can be mixed with high agitation to form a uniform aluminum hydroxide/chromium(III) oxide source mixture at room temperature (e.g., 20° C. to 40° C., preferably 25° C.). To the aluminum hydroxide/chromium(III) oxide source mixture, water, an aqueous non-metal acid (e.g., mineral acid, for example, nitric acid), the alkali metal oxide source, optionally a silica source, optionally a lanthanide oxide source, and optionally an alkaline earth metal oxide source can be added to form a formable mixture. The water, acid, alkali metal oxide source, optional silica source, optional lanthanide oxide source, and optional alkaline earth metal oxide source can be premixed and then added to the aluminum hydroxide/chromium(III) oxide source mixture, or the ingredients can be added in any order. In some embodiments, the water, mineral acid, alkali metal oxide source, optional silica source, optional lanthanide oxide source, and the optional alkaline earth metal oxide source can be premixed and the aluminum hydroxide/chromium(III) oxide source mixture can be added to the aqueous mixture. The formable mixture can be mixed at a high speed at temperature (e.g., 20° C. to 60° C., preferably 25° C.) such that the formable mixture is suitable to be formed into a shape. The formable mixture can be aged from 0.5, 1, 2, 3, 4, 5, 6, 7, 8, 9, 10, 12, 14, 16, 18, 20, 22, 24 hours or any range or value therebetween, preferably for 0.5 to 2 hours at room temperature (e.g., 20° C. to 40° C., preferably 25° C.). The formable mixture can be extruded and/or formed into any suitable shape including cylinders, cubes, stars, tri-lobes, quadra-lobes, pellets, pills, or spheres by suitable mechanical means. The shaped catalyst precursor can be heated at a temperature of 50° C. to 200° C., 100° C. to 140° C., 110° C. to 120° C., or 50° C., 55° C., 60° C., 65° C., 70° C., 75° C., 80° C., 85° C., 90° C., 95° C., 100° C., 105° C., 110° C., 115° C., 120° C., 125,° C., 130° C., 135° C., 140° C., 145° C., 150° C., or any range or value therebetween to remove water. After drying, the shaped catalyst precursor can be calcined by heating the shaped catalyst precursor to a temperature from 700° C. to 1000° C., 800° C. to 900° C., or 700° C., 725° C., 750° C., 775° C., 800° C., 825° C., 850° C., 875° C., 900° C., 925° C., 950° C., 975° C., 1000° C. or any range or value therebetween to produce the catalyst of the invention. Calcination can be performed in an inert or air atmosphere, with air atmosphere being preferred. Calcination can convert any non-oxide metal sources to oxides. For example, the non-water soluble chromium(III) oxide source can be converted to chromium oxide, an alkali metal oxide source can be converted to an alkali metal oxide (e.g., lithium oxide, sodium oxide, or mixture thereof), a lanthanide oxide source can be converted to a lanthanide oxide (e.g., cerium oxide, lanthanum oxide, or mixture thereof), and an alkaline earth metal oxide source can be converted to an alkaline earth metal oxide (e.g., barium oxide, strontium oxide, or a mixture thereof).

The formable mixture may include other conventional materials such as binders, cements, pore formers, texturizers, extrusion aids, lubricants, surfactants, and any other materials to aid with mixing or forming, or to provide a desired structure to the as-calcined material. For example, in certain embodiments, the formable mixture includes a pore forming organic compound, such as a polymer. The pore forming organic compound does not dissolve into the water of the formable mixture, and thus remains as discrete small regions of organic matter within the material when formed. During the calcination, the pore forming organic compound is burned away, which forms a gas that increases the porosity of the calcined material. The pore forming organic polymer can be, for example, a polyolefin such as polyethylene, or a cellulose derivative such as methocel. The pore forming organic compound can be provided in the formable mixture in any desirable amount, for example, in an amount within the range of about 0.1 wt. % to about 5 wt. % on a dry basis. In certain embodiments, the pore forming organic compound is present in the formable mixture in an amount within the range of about 0.2 wt. % to about 5 wt. %, or about 0.5 wt. % to about 5 wt. %, or about 0.1 wt. % to about 3 wt. %, or about 0.2 wt. % to about 3 wt. %, or about 0.5 wt. % to about 3 wt. %, or about 0.1 wt. % to about 2 wt. %, or about 0.2 wt. % to about 2 wt. %, or about 0.5 wt. % to about 2 wt. %.

The formable mixture may include an aqueous non-metal acid. The non-metal acid herein refers to an acid in which the molecular structure of the acid does not involve a metal atom. In certain embodiments of the invention, the non-metal acid is nitric acid. In other embodiments of the invention, the non-metal acid is an organic acid, such as formic acid or acetic acid. In still other embodiments of the invention, the non-metal acid is a combination of nitric acid and an organic acid such as formic acid or acetic acid. The use of an organic acid can be beneficial in that it can reduce the nitrogen oxides concentration during heat treatment; however, it can also make the peptization of alumina less efficient. A person of ordinary skill in the art will determine the appropriate amounts and types of acids to use to provide a desired formable mixture.

The raw materials used for the catalyst preparation can be mixed well in a high shear mixer followed by mixing with an aqueous non-metal acid solution until rather stiff dough/granules are obtained. This dough/granules can be extruded and/or formed into any suitable shape including cylinders, cubes, stars, tri-lobes, quadra-lobes, pellets, pills, or spheres by suitable mechanical means. In one embodiment, mixing is conducted in a high intensity environment, such as that supplied by a B&P Littleford Mixer available from B&P Littleford, 1000 Hess Avenue, Saginaw, MI 48601. In another embodiment, mixing is conducted using an Eirich Intensive Mixer, such as that supplied by Maschinenfabrik GustavEirich Gmbh & Co KG, Hardheim, Germany. Mixing is conducted for a time sufficient so that a fine uniform mix results.

### B. Materials

Solid aluminum hydroxide can be purchased from a commercial vendor. Non-limiting examples of solid aluminum hydroxide can include bayerite, nordstrandite, amorphous aluminum hydroxide, boehmite, pseudobohemite, or mixtures thereof. In some aspects, the solid aluminum hydroxide can include crystalline aluminum trihydroxides, crystalline aluminum oxide-hydroxides or gelatinous aluminum hydroxides, or a mixture thereof. Crystalline aluminum trihydroxides can be in the range of 90 wt. % to 100 wt. % of the solid aluminum hydroxides. For example, the solid aluminum hydroxide can include 90 wt. %, 91 wt. %, 92 wt. %, 93 wt. %, 94 wt. %, 95 wt. %, 96 wt. %, 97 wt. %, 98 wt. %, 99 wt. %, 100 wt. % or any value or range therebetween of crystalline aluminum trihydroxides. The solid aluminum hydroxide can include 0 wt. % to 10 wt. % of crystalline aluminum oxide-hydroxides or gelatinous aluminum hydroxides with the balance being crystalline aluminum trihydroxides. For example, the solid aluminum hydroxide can include 0 wt. %, 1 wt. %, 2 wt. %, 3 wt. %, 4 wt. %, 5 wt. %, 6 wt. %, 7 wt. %, 8 wt. %, 9 wt. %, 10 wt. %, or any range or value therebetween of crystalline aluminum oxide-hydroxides. For example, the solid aluminum hydroxide can include 0 wt. %, 1 wt. %, 2 wt. %, 3 wt. %, 4 wt. %, 5 wt. %, 6 wt. %, 7 wt. %, 8 wt. %, 9 wt. %, 10 wt. %, or any range or value therebetween of gelatinous aluminum hydroxides. In some instances, the solid aluminum hydroxide can include 90 to 99.9 wt. % of crystalline aluminum trihydroxides and 0.1 wt. % to 10 wt. % crystalline aluminum oxide-hydroxides. In another example, the solid aluminum hydroxide can include 90 to 99.9 wt. % of crystalline aluminum trihydroxides and 0.1 wt. % to 10 wt. % gelatinous aluminum hydroxides. In another example, the solid aluminum hydroxide can include 90 to 99.9 wt. % of solid crystalline aluminum trihydroxides, and either 0.1 wt. % to 10 wt. % crystalline aluminum oxide-hydroxides or 0.1 wt. % to 10 wt. % of gelatinous aluminum hydroxides. Non-limiting examples of crystalline aluminum trihydroxides can include bayerite, nordstrandite, or a mixture thereof. Non-limiting examples of crystalline aluminum oxide-hydroxides or gelatinous aluminum hydroxides or a mixture thereof can include boehmite, amorphous aluminum hydroxide, pseudobohemite, or a mixture thereof.

Non-limiting examples of water insoluble chromium(III) oxide sources can include chromium(III) oxide, and chromium(III) hydroxide, or a mixture thereof.

The alkali metal oxides sources (e.g., sodium oxide sources, lithium oxide sources, and the like) can be purchased from commercial sources. A non-limiting example of a sodium oxide sources is sodium salts. Specific examples of sodium oxide sources can include sodium acetate, sodium bicarbonate, sodium carbonate, sodium formate, sodium hydroxide, sodium metasilicate, sodium nitrate, sodium nitrite, and the like. A non-limiting example of a lithium oxide source is lithium salts. Specific examples of lithium oxide sources can include lithium acetate, lithium carbonate, lithium formate, lithium hydroxide, lithium nitrate, and the like. While not wishing to be bound by any theory, it is believed that the subsequently formed lithium oxide stabilizes defect sites within the alumina.

The alkaline earth metal oxide sources can include barium oxide sources, strontium oxide sources, or a mixture thereof. Non-limiting examples of barium oxide sources can include barium salts, and barium oxide. Specific examples of barium oxide sources can include barium oxide, barium acetate, barium bicarbonate, barium carbonate, barium formate, barium hydroxide, barium nitrate, barium nitrite, and the like. Non-limiting examples of strontium oxide sources can include strontium salts, and strontium oxide. Specific examples of strontium oxide sources can include strontium oxide, strontium acetate, strontium bicarbonate, strontium carbonate, strontium formate, strontium hydroxide, strontium nitrate, strontium nitrite, and the like.

The lanthanum oxide source can be purchased from commercial sources. Non-limiting examples of lanthanum oxide sources can include lanthanum salts, and lanthanum oxide. Specific examples of lanthanum oxide sources can include lanthanum oxide, lanthanum acetate, lanthanum bicarbonate, lanthanum carbonate, lanthanum formate, lanthanum hydroxide, lanthanum nitrate, and the like.

The silica source can be purchased from commercial sources. Non-limiting examples of the silica sources can include sodium silicate, colloidal silica, ortho silicic acid, or a mixture thereof.

### C. System and Method to Dehydrogenate Alkanes.

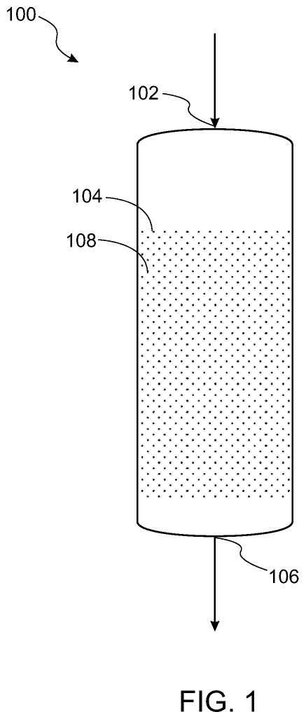

FIG. 1 depicts a schematic for a system for alkane dehydrogenation. The system 100 can include an inlet 102 for an alkane feed, a reaction zone 104 (e.g., a continuous flow reactor selected from a fixed-bed reactor, a fluidized reactor, or a moving bed reactor) that is configured to be in fluid communication with the inlet 102, and an outlet 106 that is configured to be in fluid communication with the reaction zone 104 and configured to remove a product stream from the reaction zone. Reaction zone 104 can include the dehydrogenation catalyst 108 made by the methods of the present invention. The alkane feed can enter the reaction zone 104 via the inlet 102. In some embodiments, the reactant feed stream can include inert gas (e.g., nitrogen or argon). The product stream can be removed from the reaction zone 104 via outlet 106. The product stream that includes dehydrogenated alkanes (alkenes) can be sent to other processing units, stored, and/or transported.

System 100 can include one or more heating and/or cooling devices (e.g., insulation, electrical heaters, jacketed heat exchangers in the wall) or controllers (e.g., computers, flow valves, automated values, etc.) that can be used to control the reaction temperature and pressure of the reaction mixture. While only one reactor is shown, it should be understood that multiple reactors can be housed in one unit or a plurality of reactors housed in one reactor unit. The temperature, pressure, and Gas Hourly Space Velocity (GHSV) can be varied depending on the reaction to be performed and is within the skill of a person performing the reaction (e.g., an engineer or chemist).

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Catalyst Characterization

Surface area measurement was carried out using a Micromeritics Tristar Surface Area and Porosity Analyzer. Prior to measurement, the catalyst sample (about 200 mg) was evacuated for 2 hours at 300° C. to remove physically adsorbed water and N₂ physisorption was done at −196° C.

Powder X-ray diffraction (XRD) patterns were obtained on Rigaku-Miniflex 600 X-ray diffractometer equipped with Ni-filtered CuKα (λ=1.5418Å, 40 kV, 30 mA) radiation and graphite crystal monochromator. Data was collected in the 2θ range of 29° to 41° with a step size of 0.02° and scan rate of 2°/min. The crystalline phases were identified by referring to the Joint Committee on Powder Diffraction Standards (JCPDS) database or its successor, the International Centre for Diffraction Data (ICDD).

Temperature programed reduction (TPR) experiments were carried out on an Autochem 2920 (Micromeritics) instrument. Prior to TPR analysis, a catalyst sample (about 100 mg) was pre-treated by passing pure argon (50 mL/min) at 400° C. for 30 minutes to remove physically adsorbed water. After pretreatment, the sample was cooled to 50° C., and a stream of 10% hydrogen in argon was passed through the sample and the sample heated to 600° C. at 10° C./min and the data was recorded simultaneously.

### Example 1

### Comparative Catalyst Containing Cr/Na/AI

The catalyst, with a composition of 20 wt. % Cr₂O₃, 0.40 wt. % Na₂O and 79.6 wt. % Al₂O₃ was prepared. Bayerite (2487.5 g, Pural BT, SASOL) and chromium(III) oxide (400 g, Sigma-Aldrich®) were mixed for 10 minutes in an Eirich mixer (EL-5 Profi Plus). An aqueous solution of nitric acid (462 ml, 25 wt. %) containing 21.9 g of sodium nitrate dissolved in it was added to the mixer and mixed for 10 minutes. The obtained blend was aged at 25° C. for 1 hour and then formed into cylindrical extrudates (3.5 mm diameter) using an ETP1 Bonnot lab extruder, dried at 70° C. followed by 120° C. for 12 hours, calcined at 600° C. for 2 hours in air in a muffle furnace, and cooled to room temperature without external cooling. The surface area of this catalyst was 230 m²/g. The CrO₃ content calculated from TPR was 3.3 wt. %.

### Example 2

### Catalyst of the Present Invention Containing Cr/Na/Al

The catalyst, with a composition of 20 wt. % Cr₂O₃, 0.4 wt. % Na₂O and 79.6 wt. % Al₂O₃ was prepared as in Example 1 except the sample was calcined under a nitrogen atmosphere at 850° C. The surface area of this catalyst was 139.8 m²/g. The CrO₃ content, calculated from TPR, was 1.2 wt. %.

### Example 3

### Catalyst of the Present Invention Containing Cr/Na/Al

The catalyst, with a composition of 20 wt. % Cr₂O₃, 0.4 wt. % Na₂O and 79.6 wt. % Al₂O₃ was prepared as in Example 1 except the sample was calcined in air at 850° C. The surface area of this catalyst was 89.6 m²/g. The CrO₃ content, calculated from TPR, was 1.5 wt. %.

### Example 4

### Catalyst of the Present Invention Containing Cr/Na/Li/Al

The catalyst of this example had a composition of 20 wt. % Cr₂O₃, 0.43 wt. % Na₂O, 0.25 wt. % Li₂O and 79.32 wt. % Al₂O₃. Bayerite (2408.6 g, Pural BT, SASOL), chromium(III) oxide (412 g, Sigma-Aldrich®) and pseudobohemite (128 g, PBAM-05, Chika Pvt. Ltd.) were mixed for 10 minutes in an Eirich mixer (EL-5 Profi Plus). An aqueous solution of nitric acid (476 ml, 25 wt. %) containing sodium nitrate (24.3 g) and lithium nitrate (24.3 g) dissolved in it was added to the mixer and mixed for 10 minutes. The obtained blend was aged at 25° C. for 1 hour and then formed into cylindrical extrudates (3.5 mm diameter) using an ETP1 Bonnot lab extruder, dried at 70° C. followed by 120° C. for 12 hours, calcined at 850° C. for 2 hours in air in a muffle furnace and cooled to room temperature without external cooling. The surface area of this catalyst was 90.2 m²/g. The CrO₃ content, calculated from TPR, was 1.5 wt. %.

### Example 5

### Catalyst of the Present Invention Containing Cr/Na/Li/Al

The catalyst, with a composition of 20 wt. % Cr₂O₃, 0.43 wt. % Na₂O, 0.25 wt. % Li₂O and 79.32 wt. % Al₂O₃ was prepared in the same manner as Example 4 except that the calcination was performed at 900° C. The surface area of this catalyst was 60.8 m²/g. The CrO₃ content, calculated from TPR, was 1.2 wt. %.

### Example 6

### Catalyst of the Present Invention Containing Cr/Na/Li/Ba/AI

The catalyst, with a composition of 20 wt. % Cr₂O₃, 0.43 wt. % Na₂O, 0.25 wt. % Li₂O, 1.78 wt. % BaO and 77.54 wt. % Al₂O₃ was prepared. Bayerite (2286.1 g, Pural BT, SASOL), chromium(III) oxide (400 g, Sigma-Aldrich®), pseudoboehmite (121.4 g, PBAM-05, Chika Pvt. Ltd.) and barium nitrate (60.8 g) were mixed for 10 minutes in an Eirich mixer (EL-5 Profi Plus). An aqueous solution of nitric acid (463 ml, 25 wt. %) containing sodium nitrate (23.6 g) and lithium nitrate (22.8 g) dissolved in it was added to the mixer and mixed for 10 minutes. The obtained blend was aged at 25° C. for 1 hour and then formed into cylindrical extrudates (3.5 mm diameter) using an ETP1 Bonnot lab extruder, dried at 70° C. followed by 120° C. for 12 hours, calcined at 850° C. for 2 hours in air in a muffle furnace and cooled to room temperature without external cooling. The surface area of this catalyst was 90.7 m²/g. The CrO₃ content, calculated from TPR, was 2.1 wt. %.

### Example 7

### Comparative Catalyst Containing Cr/Na/Li/Ba/AI

The comparative catalyst, with a composition of 20 wt. % Cr₂O₃, 0.43 wt. % Na₂O, 0.25 wt. % Li₂O, 1.78 wt. % BaO and 77.54 wt. % Al₂O₃ was prepared. Bayerite (2829.1 g, Pural BT, SASOL), pseudoboehmite (148.9 g, PBAM-05, Chika Pvt. Ltd.) and barium nitrate (76.24 g) was mixed for 10 minutes in an Eirich mixer (EL-5 Profi Plus). An aqueous solution of nitric acid (500 ml, 15 wt. %) containing lithium nitrate (28.59 g) dissolved in it was added to the mixer and mixed for 10 minutes. The obtained blend was aged at 25° C. for 1 hour and then formed into cylindrical extrudates (3.5 mm diameter) using an ETP1 Bonnot lab extruder, dried at 70° C. followed by 120° C. for 12 hours, calcined at 850° C. for 2 hours in air in a muffle furnace and cooled to room temperature without external cooling. The calcined alumina extrudates (250 g) were impregnated to incipient wetness with an aqueous solution containing chromium (VI) oxide (78 g) and sodium dichromate dihydrate (7 g). The wet extrudates were aged 25° C. for 12 hours in a closed container. The sample was dried for 6 hours at 120° C. and calcined at 750° C. for 2 hours in air in a muffle furnace and cooled to room temperature without external cooling. The surface area of this catalyst was 83.6 m²/g. The CrO₃ content, calculated from TPR, was 2.5 wt. %.

### Example 8

### Catalyst of the Present Invention Containing Cr/Na/La/Al

The catalyst of the present invention, with a composition of 25.2 wt. % Cr₂O₃, 1 wt. % Na₂O, 0.5 wt. % La₂O₃ and 73.3 wt. % Al₂O₃ was prepared. Bayerite (2267.13 g. Pural BT, SASOL), chromium(III) oxide (527.62 g, Sigma-Aldrich®) and pseudoboehmite (119.32 g, PBAM-05, Chika Pvt. Ltd.) were mixed for 10 minutes in an Eirich mixer (R-02). An aqueous solution of nitric acid (660 ml, 25 wt. %) containing sodium nitrate (57.88 g) and lanthanum nitrate hexahydrate (28.05 g) dissolved in it was added to the mixer and mixed for 18 minutes. The obtained blend was aged at 25° C. for 1 hour and then formed into cylindrical extrudates (3.5 mm diameter) using an ETP-1 Bonnot like lab extruder, dried at 70° C. followed by 120° C. for 12 hours, calcined at 800° C. for 2 hours in air in a muffle furnace in air and cooled to room temperature without external cooling.

### Example 9

### Catalyst of the Present Invention Containing Cr/Na/La/Al

The catalyst of the present invention, with a composition of 25 wt. % Cr₂O₃, 1 wt. % Na₂O, 1 wt. % La₂O₃ and 73 wt. % Al₂O₃ was prepared using the method of Example 8 except that the amounts of the various ingredients/components were modified. Specifically, Example 9 used the following amounts of each ingredient: 2246.13 g Bayerite (Pural BT, SASOL), 522.73 g chromium(III) oxide, 118.22 g pseudobohemite, 550 mL nitric acid, 57.35 g sodium nitrate, and 55.58 g lanthanum nitrate hexahydrate.

### Example 10

### Catalyst of the Present Invention Containing Cr/Na/La/AI

The catalyst of the present invention, with a composition of 24.5 wt. % Cr₂O₃, 1 wt. % Na₂O, 2.9 wt. % La₂O₃ and 71.5 wt. % Al₂O₃ was prepared using the method of Example 8 except the amounts of the various ingredients/components were modified. Specifically, Example 10 used the following amounts of each ingredient: 2165.88 g Bayerite, 504.05 g chromium(III) oxide, 113.99 g pseudobohemite, 616 mL nitric acid, 55.30 g sodium nitrate and 160.78 g lanthanum nitrate hexahydrate.

### Example 11

### Catalyst of the Present Invention Containing Cr/Na/La/Ba/Al

The catalyst of the present invention, with a composition of 25 wt. % Cr₂O₃, 1.0 wt. % Na₂O, 0.74 wt. % BaO and 0.89 wt. % La₂O₃ and 72.4 wt. % Al₂O₃ was prepared. Bayerite (2134.4 g, Pural BT, SASOL), chromium(III) oxide (500 g, Sigma-Aldrich®), pseudoboehmite (112.7 g, PBAM-05, Chika Pvt. Ltd.), and barium nitrate (25.2 g) were mixed for 10 minutes in an Eirich mixer (EL-5 Profi Plus). An aqueous solution of nitric acid (463 ml, 25 wt. %) containing sodium nitrate (54.9 g) and lanthanum nitrate hexahydrate (47.2 g) dissolved in it was added to the mixer and mixed for 10 minutes. The obtained blend was aged at 25° C. for 1 hour and then formed into cylindrical extrudates (3.5 mm diameter) using an ETP1 Bonnot lab extruder, dried at 70° C. followed by 120° C. for 12 hours, calcined at 800° C. for 2 hours in air in a muffle furnace and cooled to room temperature without external cooling. The surface area of this catalyst was 94.9 m²/g.

### Example 12

### Comparative Catalyst Containing Cr/Na/La/Al

The comparative catalyst, with a composition of 25 wt. % Cr₂O₃, 1.0 wt. % Na₂O, 3 wt. % La₂O₃ and 71 wt. % Al₂O₃ was prepared. Bayerite (2850 g, Pural BT, SASOL) and pseudoboehmite (150 g, PBAM-05, Chika Pvt. Ltd.) were mixed for 10 minutes in an Eirich mixer (R-02). An aqueous solution of nitric acid (580 ml, 20 wt. %) was added to the mixer and mixed for 15 minutes. The obtained blend was aged at 25° C. for 1 hour and then formed into cylindrical extrudates (3.5 mm diameter) using an ETP1 Bonnot like lab extruder, dried at 70° C. followed by 120° C. for 12 hours, calcined at 550° C. for 2 hours in air in a muffle furnace and cooled to room temperature without external cooling. The prepared alumina extrudates (96 g) were impregnated to incipient wetness with an aqueous solution containing lanthanum nitrate hexahydrate (10.63 g). The wet extrudates were aged at 25° C. for 12 hours in a closed container. The sample was then dried for 6 hours at 120° C. and calcined at 800° C. for 2 hours in air in a muffle furnace and cooled to room temperature without external cooling. The calcined modified alumina extrudates (74 g) were impregnated to incipient wetness with an aqueous solution containing chromium (VI) oxide (29.67 g) and sodium dichromate dihydrate (4.81 g). The wet extrudates were aged at 25° C. for 12 hours in a closed container. The sample was then dried for 6 hours at 120° C. and calcined at 750° C. for 2 hours in air in a muffle furnace and cooled to room temperature without external cooling.

### Example 13

### Comparative Catalyst Containing Eta Alumina and Cr/Na/La

The alumina carrier with eta-alumina used for the catalyst preparation was prepared. Bayerite (3125 g, Pural BT, SASOL) was mixed for 10 minutes in an Eirich mixer (EL-5 Profi Plus). An aqueous solution of nitric acid (520 ml, 15 wt. %) was added to the mixer and mixed for 9 minutes. The obtained blend was aged at 25° C. for 1 hour and then formed into cylindrical extrudates (3.5 mm diameter) using an ETP1 Bonnot lab extruder, dried at 70° C. followed by 120° C. for 12 hours, calcined at 600° C. for 2 hours in air in a muffle furnace and cooled to room temperature without external cooling.

Using this alumina carrier, the comparative catalyst, with a composition of 25 wt. % Cr₂O₃, 1.0 wt. % Na₂O, 1 wt. % La₂O₃ and 73 wt. % Al₂O₃ was prepared. The calcined alumina extrudates (73 g) were impregnated to incipient wetness with an aqueous solution containing chromium (VI) oxide (9.7 g), lanthanum oxide (1 g) and sodium dichromate dihydrate (2.5 g). The wet extrudates were aged 25° C. for 12 hours in a closed container. The sample was dried for 6 hours at 120° C. and calcined at 750° C. for 2 hours in air in a muffle furnace and cooled to room temperature without external cooling. The surface area of this catalyst was 88.8 m²/g.

### Example 14

### Comparative Catalyst Containing Eta and Gamma Alumina and Cr/Na/La

The alumina carrier with 95 wt. % eta-alumina and 5 wt. % gamma-alumina used for the catalyst preparation was prepared. Bayerite (2969 g, Pural BT, SASOL) and pseudoboehmite (139 g, PBAM-05, Chika Pvt. Ltd.) were mixed for 10 minutes in an Eirich mixer (EL-5 Profi Plus). An aqueous solution of nitric acid (520 ml, 15 wt. %) was added to the mixer and mixed for 8.5 minutes. The obtained blend was aged at 25° C. for 1 hour and then formed into cylindrical extrudates (3.5 mm diameter) using an ETP1 Bonnot lab extruder, dried at 70° C. followed by 120° C. for 12 hours, calcined at 600° C. for 2 hours in air in a muffle furnace and cooled to room temperature without external cooling.

The comparative catalyst using the alumina extrudate, having a composition of 25 wt. % Cr₂O₃, 1.0 wt. % Na₂O, 1 wt. % La₂O₃ and 73 wt. % Al₂O₃ was prepared. The calcined alumina extrudates (73 g) were impregnated to incipient wetness with an aqueous solution containing chromium (VI) oxide (29.7 g), lanthanum oxide (1 g) and sodium dichromate dihydrate (2.5 g). The wet extrudates were aged 25° C. for 12 hours in a closed container. The sample was dried for 6 hours at 120° C. and calcined at 750° C. for 2 hours in air in a muffle furnace and cooled to room temperature without external cooling. The surface area of this catalyst was 94.9 m²/g.

### Example 15

### Analysis

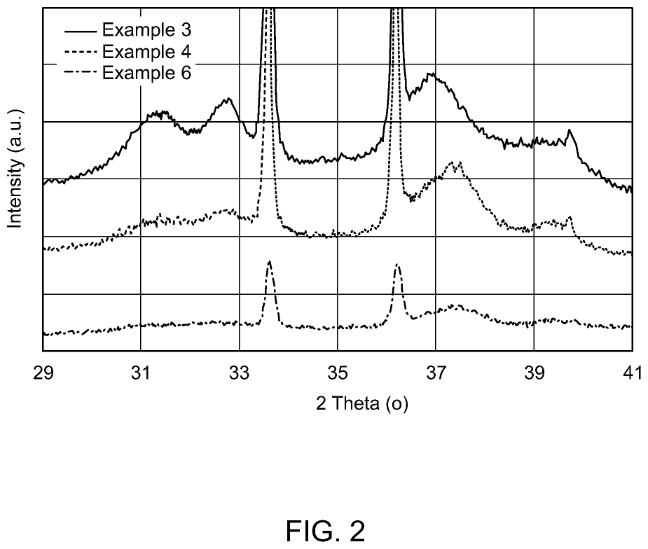

Powder XRD patterns of catalysts of Example 3, Example 4 and Example 6 are shown in FIG. 2. The top diffraction pattern is a catalyst of the present invention that includes 20 wt. % Cr₂O₃, 0.40 wt. % Na₂O and 79.6 wt. % Al₂O₃ and calcination was performed in air at 850° C. The middle diffraction pattern is a catalyst of the present invention that includes 20 wt. % Cr₂O₃, 0.43 wt. % Na₂O, 0.25 wt. % Li₂O and 79.32 wt. % Al₂O₃, the Al₂O₃ is from bayerite and pseudobohemite, and was calcination was performed in air at 850° C. The bottom diffraction pattern is a catalyst of the present invention that includes 20 wt. % Cr₂O₃, 0.43 wt. % Na₂O, 0.25 wt. % Li₂O, 1.78 wt. % BaO and 77.54 wt. % Al₂O₃, the Al₂O₃ is from bayerite and pseudobohemite and calcination was performed in air at 850° C. In the Example 3 catalyst of the present invention, the alumina existed mainly in theta alumina form, the Example 4 catalyst of the present invention had a low theta alumina form, and Example 6 catalyst of the present invention, the alumina had no theta alumina. Thus, from these results, it was determined that the presence of (a) lithium oxide in the catalyst of Example 4, and (b) lithium oxide and barium oxide in catalyst of Example 6, stabilized the alumina against phase transformation. Thus, catalyst of Example 6 of the present invention showed higher thermal stability in comparison with catalysts of Example 3 and 4. The catalyst with higher thermal stability is expected to have higher stability in isobutane dehydrogenation.

### Example 16

### Catalyst Testing

The dehydrogenation activity of the prepared catalyst was measured in a tubular fixed-bed quartz reactor under atmospheric pressure. Catalyst loading and reactor details were as follows: Catalyst weight=8.5 g, catalyst particle size=~3 mm, inert quartz weight=8.5 g, inert quartz chips=0.4-0.5 mm, reactor inside diameter=16 mm, reactor outside diameter=19 mm. Catalyst and inert quartz were divided into equal parts by weight and then loaded into the reactor by mixing catalyst and inert. Isobutane (99.9 vol. %) was used as the feed. Quartz chips having a size of 1-1.4 mm were loaded above the catalyst bed. A nitrogen purge was employed between the steps of dehydrogenation, catalyst regeneration/oxidation and reduction with hydrogen. The total feed flow in the dehydrogenation step corresponds to a GHSV=600 mL h⁻¹ g⁻¹. The reactor outlet gases were analyzed by an online gas chromatograph (Agilent 6890) equipped with a flame ionization detector for hydrocarbon analysis and a thermal conductivity detector for hydrogen analysis. The reactant and products flow rates were measured using a Ritter type wet gas flow meter. The reactor was operated at atmospheric pressure and in a cyclic mode with the following steps: 1) catalyst oxidation with air with a start temperature of 650° C. for 20 min.; 2) purge the catalyst with nitrogen at 650° C. for 3 min.; 3) reduce the catalyst with H₂ with a start temperature of 650° C. for 6 min.; 4) cool under nitrogen from 650° C. to 585° C. and maintaining a temperature of 585° C. for 30 min.; 5) dehydrogenation of isobutane with a start temperature of 585° C. for 21 min.; 6) analyze the reactor outlet gas composition with gas chromatograph (GC) at the 20th minute from the start of the isobutane feed. Steps 1 to 6 were repeated for 30 times. The catalyst performance data (10 cycle average, with standard deviations) after catalyst stabilization is given in Table 1 and 2.

### Example 17

### Catalyst Testing

Catalyst stability evaluation was carried out by an artificial accelerated aging procedure in a cyclic mode of operation. The cycle included H₂—N₂-isobutane-N₂-air flow stages with different time durations. The aging was carried out at 820° C. for 72 hours. Catalyst stability evaluation parameters included a catalyst weight=8.5 g, isobutane GHSV=400 mL h⁻¹ g⁻¹ and air to isobutane volume ratio of 2. The following steps were performed: 1) oxidize the catalyst under air for 15 min.; 2) purge the catalyst with nitrogen for 3 min.; 3) reduce the catalyst with H₂ for 6 min.; 4) purge the catalyst with nitrogen for 3 min.; 5) isobutane flow for 3 min.; and 6) purge the catalyst with nitrogen for 3 min. After aging, catalyst performance was carried out under the cyclic mode mentioned previously.

The performance of catalysts are listed in Table 1 and 2. The results from Table 1 shows that catalyst prepared by calcination at 850° C. in air shows higher isobutylene selectivity in comparison with catalyst calcined at 600° C. in air. Similarly, the catalyst prepared by calcination at 850° C. in air shows higher isobutylene selectivity in comparison with catalyst calcined at 850° C. in nitrogen.

TABLE 1

| | Conversion (mol %) | | Selectivity (mol %) | | Yield (mol %) | |
|---|---|---|---|---|---|---|
| Example | Aver. | SD | Aver. | SD | Aver. | SD |
| 1 | 50.0 | 0.6 | 82.4 | 1.3 | 41.2 | 0.8 |
| 2 | 50.3 | 1.0 | 85.0 | 1.0 | 42.7 | 0.9 |
| 3 | 52.0 | 0.2 | 90.3 | 0.3 | 46.9 | 0.1 |
| 5 | 53.5 | 0.3 | 92.0 | 0.9 | 49.2 | 0.2 |

TABLE 2

| | Conversion (mol %) | | | | Selectivity (mol %) | | | | Yield (mol %) | | | |
|---|---|---|---|---|---|---|---|---|---|---|---|---|
| | Before | | After | | Before | | After | | Before | | After | |
| | aging | | aging | | aging | | aging | | aging | | aging | |
| Example | Aver. | SD | Aver. | SD | Aver. | SD | Aver. | SD | Aver. | SD | Aver. | SD |
| 6 | 51.3 | 0.3 | 48.5 | 0.4 | 92.7 | 1.0 | 91.8 | 1.3 | 47.5 | 0.4 | 44.5 | 0.3 |
| 7 (com) | 51.4 | 1.6 | 46.0 | 0.7 | 92.7 | 1.7 | 93.0 | 1.7 | 47.6 | 0.7 | 42.8 | 0.4 |
| 8 | 52.7 | 0.4 | 50.0 | 0.5 | 93.4 | 1.1 | 95.0 | 1.5 | 49.2 | 0.3 | 47.0 | 0.4 |
| 9 | 52.5 | 0.2 | 48.5 | 0.4 | 95.6 | 1.1 | 93.7 | 1.4 | 50.2 | 0.5 | 45.4 | 0.4 |
| 10 | 53.1 | 0.3 | 49.4 | 0.8 | 94.1 | 1.0 | 93.1 | 1.6 | 50.0 | 0.3 | 46.0 | 0.3 |
| 11 | 53.3 | 0.3 | 48.0 | 0.6 | 94.5 | 1.1 | 94.0 | 1.8 | 50.4 | 0.3 | 45.1 | 0.3 |
| 12 (com) | 54.0 | 0.4 | 44.4 | 0.3 | 92.4 | 1.2 | 92.0 | 0.8 | 49.9 | 0.3 | 40.8 | 0.2 |
| 13 (com) | 54.8 | 0.4 | 45.0 | 0.6 | 94.0 | 0.9 | 91.9 | 1.1 | 51.5 | 0.5 | 41.4 | 0.5 |
| 14 (com) | 54.4 | 0.4 | 43.0 | 0.3 | 91.4 | 1.2 | 92.2 | 1.3 | 49.8 | 0.4 | 39.6 | 0.3 |

Further, when comparing the performance of Example 6 catalyst of the present invention vs Example 7 comparative catalyst, Example 10 catalyst of the present invention vs Example 12 comparative catalyst, and Example 9 catalyst of the present invention vs Examples 13 and 14 comparative catalysts, it was determined that catalysts prepared by the method of the present invention showed higher stability compared to the catalyst prepared according to the comparative Examples.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1. A method of preparing an alkane dehydrogenation catalyst, the method comprising:
(a) extruding and/or tableting a formable mixture comprising a solid aluminum hydroxide, a water insoluble chromium(III) oxide source, an alkali metal oxide source, and a non-metal acid, to produce a catalyst precursor;
(b) drying the catalyst precursor; and
(c) calcining the dried catalyst precursor at a temperature of 700° C. to 1000° C. to produce an alkane dehydrogenation catalyst comprising 10 wt. % to 40 wt. % chromium(III) oxide (Cr₂O₃), 0.1 wt. % to 5 wt. % alkali metal oxide, and 60 wt. % to 90 wt. % alumina (Al₂O₃).

2. The method of claim 1, further comprising calcining the catalyst in the presence of air.

3. The method of claim 1, wherein the alkali metal oxide source comprises a sodium oxide source, a lithium oxide source, a cesium oxide source, a potassium oxide source, or a mixture thereof.

4. The method of claim 3, wherein the catalyst comprises:
60 wt. % to 90 wt. % Al₂O₃;
10 wt. % to 40 wt. % Cr₂O₃; and
0.1 wt. % to 5 wt. % Na₂O.

5. The method of claim 4, wherein the catalyst comprises:
60 wt. % to 90 wt. % Al₂O₃;
10 wt. % to 40 wt. % Cr₂O₃;
0.1 wt. % to 5 wt. % Na₂O; and
0.1 wt. % to 5 wt. % Li₂O.

6. The method of claim 1, wherein the formable mixture further comprises an alkaline earth metal oxide source and the catalyst comprises 0.1 wt. % to 20 wt. % alkaline earth metal oxide source, wherein the alkaline earth metal oxide source comprises a barium oxide source (BaO) and/or a strontium oxide source (SrO), and the catalyst comprises BaO, SrO, or a combination thereof.

7. The method of claim 1, wherein the catalyst comprises:
60 wt. % to 90 wt. % Al₂O₃;
10 wt. % to 40 wt. % Cr₂O₃;
0.1 wt. % to 5 wt. % Na₂O;
0.1 wt. % to 20 wt. % BaO; and
optionally 0.1 wt. % to 5 wt. % Li₂O.

8. The method of claim 1, wherein the formable mixture further comprises lanthanide oxide source, the lanthanide oxide source comprising a lanthanum oxide source, a cerium oxide source, or a mixture thereof, and the catalyst comprises 0.1 wt. % to 20 wt. % lanthanum oxide (La₂O₃).

9. The method of claim 8, wherein the catalyst comprises;
60 wt. % to 95 wt. % Al₂O₃;
5 wt. % to 40 wt. % Cr₂O₃;
0.1 wt. % to 5 wt. % Na₂O; and
0.1 wt. % to 20 wt. % La₂O₃.

10. The method of claim 8, wherein the formable mixture further comprises an alkaline earth metal oxide source and the catalyst comprises 0.1 wt. % to 20 wt. % alkaline earth metal oxide, wherein the alkaline earth metal oxide source comprises a barium oxide source and/or strontium oxide source and the catalyst comprises BaO, SrO, or a combination thereof.

11. The method of claim 10, wherein the catalyst comprises:
60 wt. % to 90 wt. % Al₂O₃;
10 wt. % to 40 wt. % Cr₂O₃;
0.1 wt. % to 5 wt. % Na₂O;
0.1 wt. % to 20 wt. % BaO; and
0.1 wt. % to 20 wt. % La₂O₃.

12. The method of claim 1, wherein the formable mixture further comprises a silica source, and the catalyst comprises 0.1 wt. % to 5 wt. % silica.

13. The method of claim 1, wherein the solid aluminum hydroxide comprises crystalline aluminum trihydroxides, and wherein the crystalline aluminum trihydroxides comprise bayerite, nordstrandite, or a mixture thereof.

14. The method of claim 1, wherein the solid aluminum hydroxide comprises crystalline aluminum trihydroxides, crystalline aluminum oxide-hydroxides, gelatinous aluminum hydroxides, or a mixture thereof, wherein the crystalline aluminum trihydroxides comprise bayerite, nordstrandite, or a combination thereof, wherein the crystalline aluminum oxide hydroxides comprise boehmite, and wherein the gelatinous aluminum hydroxides comprise amorphous aluminum hydroxide, pseudobohemite, or a combination thereof, and wherein the water insoluble chromium(III) oxide sources comprise chromium(III) oxide, chromium(III) hydroxide, or a combination thereof.

15. A method to dehydrogenate alkanes comprising contacting a catalyst made by the method of claim 1 with an alkane composition to produce dehydrogenated alkanes.
